(19) United States
(12) Patent Application Publication
(10) Pub. No.: US 2005/0070012 A1
(43) Pub. Date: Mar. 31, 2005

(54) Cartridge for biochemical analysis unit

(75) Inventor: Katsuaki Muraishi, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/950,819

(22) Filed: Sep. 28, 2004

(30) Foreign Application Priority Data
Sep. 29, 2003 (JP) 2003-338239

(51) Int. Cl. C12M 1/34

(52) U.S. Cl. 435/287.2; 435/297.2; 422/101; 422/102; 435/288.4

(57) ABSTRACT

A cartridge includes a cartridge main body and a filter unit. The cartridge main body contains a biochemical analysis unit of flow-through type, and includes a chamber into which a reaction solution is injected. The filter unit is used for the filtrating the reaction solution and fixed by screws to a bottom of the cartridge main body. The biochemical analysis unit is removably loaded onto a reactor main body in a situation that the cartridge is loaded. In setting the cartridge, the biochemical analysis unit and the filter are loaded, and therefore the loading mistake is prevented and the time for exchange of the filter is reduced.

### BACKGROUND OF THE INVENTION

1. Field of the Invention

The present invention relates to a cartridge for containing a biochemical analysis unit that is used for base sequence analysis of DNA and the like.

2. Description Related to the Prior Art

In order to make a biochemical analysis for base sequence of substances derived from living organism (for example DNA), a biochemical analysis unit is used. In order to obtain the biochemical analysis unit, minute through holes are formed in the substrate, and porous materials and the like are pressed into each through hole to form a spot area. Thus, the spot areas are arranged on the substrate, and therefore the biochemical analysis unit is called also microarray. A method of biochemical analysis, in which the biochemical analysis unit is used, includes a spotting process, a reaction process, a data reading process, and a data analysis process. In the spotting process, a specific binding substance as a reagent (hereinafter probe) is spotted and fixed in the spot areas on the biochemical analysis unit. In the reaction process, a specific binding substance as a test body (hereinafter target) is penetrated into the spot areas, and the specific binding (the binding between the probe and the target) is made. In the data reading process a biochemical analysis data is read out from the biochemical analysis unit as a result of the specific binding reaction in each spot area. In the data analysis process, the read out analysis data is analyzed in the personal computer and the like. (see, Japanese Patent Laid-Open Publication No. 2003-227825, and International Publication under PCT No. 01/45843).

Since the probe is a reagent for searching the information of expression, the molecular structure (for example base sequence, composition and the like) of the used probe is already known. As the probe, there are hormones, tumor markers, enzymes, antibodies, antigens, abzymes, receptors, other proteins, ligand, nucleic acids, cDNA, DNA, RNA, and the like, and the probe can make a specific binding to the target, whose molecular structure is not known. As the target, there are substances derived from living organism (such as hormones, tumor markers, enzymes, antibodies, antigens, abzymes, receptors, other proteins, ligand, nucleic acids, cDNA, DNA, mRNA, and the like, which are extracted and isolated from the living organism), and products obtained by performing the chemical treatments or the chemical modifications of the substances derived from living organism.

When the base sequence is searched, several sorts of the probes are fixed in respective spot areas of the biochemical analysis unit. Then in the reaction process, a solution in which the target is dissolved to a solvent is penetrated in the spot areas, and the specific binding of the target and the probe having a complementary relation to the target is made.

In order to detect the specific binding, the reaction solution contains for example a labeling substances. As the labeling substances to be used, there are fluorescent substances which generate a chemical fluorescence in a chemical reaction. After the specific binding is made, the biochemical analysis unit is cleaned to remove the reaction solution on other areas than spot areas.

In the spot area in which the specific binding is made, the labeling substances remain. Accordingly, in the data reading process, a labeling signal of the optical ray, the radioactive ray or the like generated from the labeling substances is read for detecting the specific binding. As the detecting device to be used, there is an imaging device, for example, a CCD imaging sensor for reading the optical information.

In order to make the specific binding reaction, the reaction solution is injected in a reaction chamber in which the biochemical analysis unit is contained, and then a piston, a pump and the like are driven to flow the reaction solution from one side to another side of the spot are with a mechanical pressure. This method is usually made and called a flow through method. (See, WO 01/45843)

Further, recently, in consideration with the easiness of the treatment of the biochemical analysis unit, a flow through type cartridge with chamber for containing the biochemical analysis unit is developed. When this cartridge is used, it becomes unnecessary to load or unload a naked biochemical analysis unit in or from a reactor. The reactor corresponding to the cartridge type has a reactor main body for loading the cartridge and a circulating device, such as a piston and a circle pipe, for circulating the reaction solution. The circulating device is connected to a loading section. Only when the cartridge is loaded, the circulating device and the chamber are connected.

However, in the biochemical analysis unit of the flow through type, foreign materials such as undisolved materials and precipitations in the reaction solution clogs the spot areas. The clogging causes a noise when the data reading is made, and in this case the accuracy of data analysis becomes lower. Accordingly, in the reactor described in the publication No. 2003-227825, a filter for filtrating the reaction solution is provided in the circulation passage to remove the undissolved materials and the precipitations from the reaction solution supplied into the chamber.

However, it is necessary to exchange the filter and the cartridge for each experiment. When the number of members to be exchanged becomes larger, the time for labor becomes larger for the exchange. Further, the loading is sometimes made badly or forgot, and the mistakes of the loading or the exchange are sometimes made.

### SUMMARY OF THE INVENTION

An object of the present invention is to provide a biochemical analysis cartridge for preventing mistakes of loading the filter and making the exchanging time shorter.

In order to achieve the object and other objects, a cartridge for containing a biochemical analysis unit of flow through type with arrangement of plural spot areas is removably contained in a reactor. Probes are reagents are fixed to the spot areas, and a target as a test substance in a reaction solution specifically reacts to the probes. The cartridge includes a cartridge main body for removably containing the biochemical analysis unit, and a chamber formed in the cartridge main body. The chamber has an inlet for supplying the reaction solution thereof and on outlet for discharging the reaction solution. The biochemical analysis unit is positioned so as to partition the chamber into a supply part and a discharge part. The cartridge is provided with a filter disposed upstream from the inlet, so as to filter the reaction solution.

The cartridge further includes a filter case for containing the filter. The filer case is integrated with the cartridge main body.

The cartridge for the biochemical analysis unit of the present invention is removably set to the reactor, and includes the cartridge main body and the filter for filtering the reaction solution to be supplied to the chamber in the cartridge main body. Since the filter is fixedly positioned to the cartridge main body, the loading failure of the filter is prevented and the time for exchange is reduced.

### BRIEF DESCRIPTION OF THE DRAWINGS

The above objects and advantages of the present invention will become easily understood by one of ordinary skill in the art when the following detailed description would be read in connection with the accompanying drawings.

FIG. 1 is a flow chart illustrating all processes of biochemical analyzing method in which biochemical analysis unit is used;

FIG. 2A is a plan view of a biochemical analysis unit;

FIG. 2B is an exploded plan view of a biochemical analysis unit;

FIG. 3 is a sectional view of a cartridge and a reactor;

FIG. 4 is an exploded perspective view of the cartridge;

FIG. 5 is an exploded perspective view of a reactor main body.

### PREFERRED EMBODIMENTS OF THE INVENTION

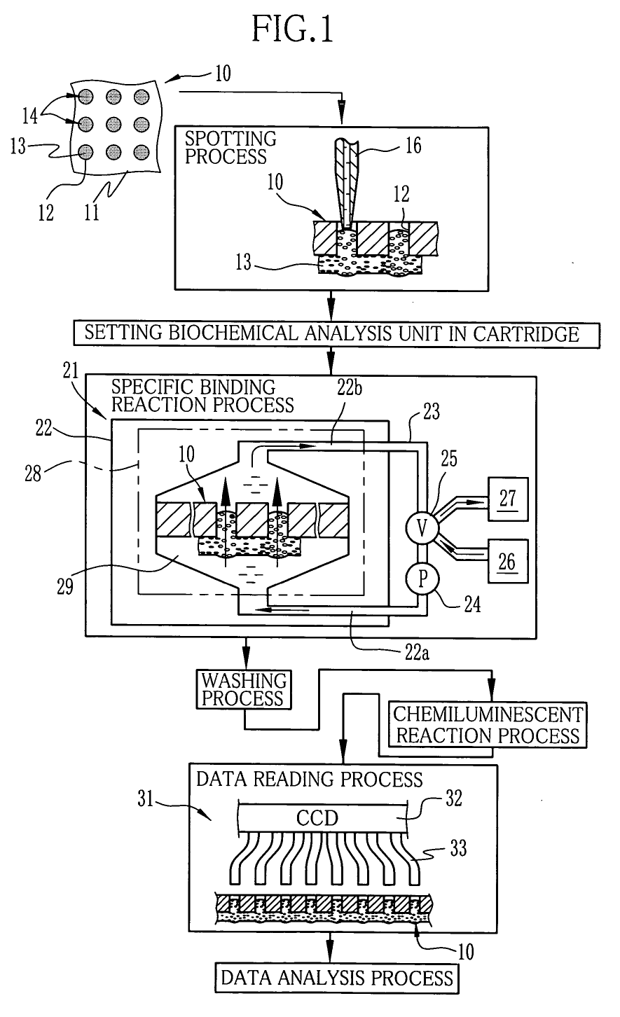

As shown in FIG. 1, a biochemical analyzing method in which a biochemical analysis unit 10 is used includes a spotting process, a specific binding reaction process, a cleaning process, a chemiluminescence reaction process, a data reading process and a data analysis process. In the biochemical analysis unit 10, minute through holes 12 are formed in matrix-arrangement in the substrate 11, and a membrane 13 of the absorptive material is pressed into the through holes 12. Thus a spot area 14 are formed in each through hole 12, and the obtained biochemical analysis unit is a flow-through type.

In the spotting process, solutions containing different probes (hereinafter probe solutions) are spotted in the respective spot area 14 of the biochemical analysis unit 10 with use of a spotter. The spotter has a spot pin 16, of which a groove is formed on a tip for spotting the prove solution. Several kinds of the probe solutions dispensed on a well plate are sucked up and spotted in the spot area 14 by the spot pins 16. Thereafter, an UV-ray is irradiated on the spot areas 14 to fix the probe therein. Thus the biochemical analysis unit 10 in which the probes are fixed is placed in a biochemical analysis cartridge (hereinafter cartridge) 28 including a chamber 29. Then the biochemical analysis unit 10 is transported to the specific binding reaction process.

In the specific binding reaction process, the specific binding reaction of the probes and a target as the test substance is made. The reactor 21 is constructed of a reactor main body 22, a circulating pipe 23, a pump 24, a valve 25 a solution tank 26, a discharge vessel 27, and the like. The cartridge 28 is removably set to the reactor main body 22. In order to prepare the reaction solution, the target bound to the labeling materials is dissolved to the solvent.

The circulation pipe 23 composes a circulation mechanism along with the pump 24 and the valve 25. The reactor main body 22 is provided with a supply path 22a and a discharge path 22b which are connected to the chamber 29. The supply path 22a and the discharge path 22b are combined with the circulating pipe 23 to construct a circulation passage for the reaction solution flowing through the chamber 29. Further, the solution tank 26 in which the unused solution is contained and the discharge vessels 27 in which the used solution is contained are connected through the valve 25 to the circulating pipe 23.

The reaction solution injected into the chamber 29 penetrates into the spot areas 14 from a lower side to an upper side in the figure. Thereby, in some spot areas 14 in which the probes having the complementary relation to the target are fixed, the specific binding of the target and the probe is made. Then the reaction solution flows through the spot areas 14 and is discharged through a discharge opening 29b from the chamber 29. Thereafter, the reaction solution discharged from the chamber 29 is sent through the circulating pipe 23 and the pump 24 to an entrance 29a, and supplied into the chamber 29 again.

In the cleaning process after the specific binding reaction process, the biochemical analysis unit 10 is cleaned and the reaction solution is removed from other area than the some spotting areas in which the specific binding is made. In this cleaning process, the reactor 21 is used as it is in the specific binding reaction process. In the chamber 29, a cleaning solution is supplied instead of the reaction solution in the specific binding reaction process, and the cleaning is made in the flowage of the cleaning solution. Preferably the blocking agent is penetrated into the biochemical analysis unit in advance of making the reaction. In this case, the target which has not made the specific binding reaction can be easily removed, and thus the effect of the cleaning becomes better. In the penetration of the blocking agent into the biochemical analysis unit 10, it is preferable to use the reactor 21 in the same manner as the cleaning solution.

After the cleaning process, the chemiluminescent reaction is made. In the chemiluminescent reaction process, the spot area in which specific binding is made generates a luminescence.

After the chemiluminescent reaction process, the cartridge 28 is removed from the reactor main body 22 and sent to the data reading process. In the data reading process, the biochemical analysis unit 10 is removed from the cartridge 28, and the biochemical analysis data is photoelectrically read by a detecting device 31.

The detecting device 31 includes a CCD image sensor 32 which receives a light generated from the labeling substances and photoelectrically converts the light. In front of a receiving surface of the CCD image sensor 32, there is a light guide for guiding the light to photosensitive elements of the CCD image sensor 32. A light guide 33 is constructed of optical fibers whose number is corresponding to that of the spot areas 14. One end of each optical fiber confronts to the receiving surface and another end to the corresponding spot area 14. Since the labeling substances remain in the some spot areas in which the specific binding reaction is made, the light is generated. Otherwise, the light is not generated in the other spot areas in which the specific binding is not made. An image data formed as the result of the specific binding reaction in the spot areas 14 is received by the CCD image sensor 32. In the data analyzing process, the image data is analyzed as the biochemical analysis data.

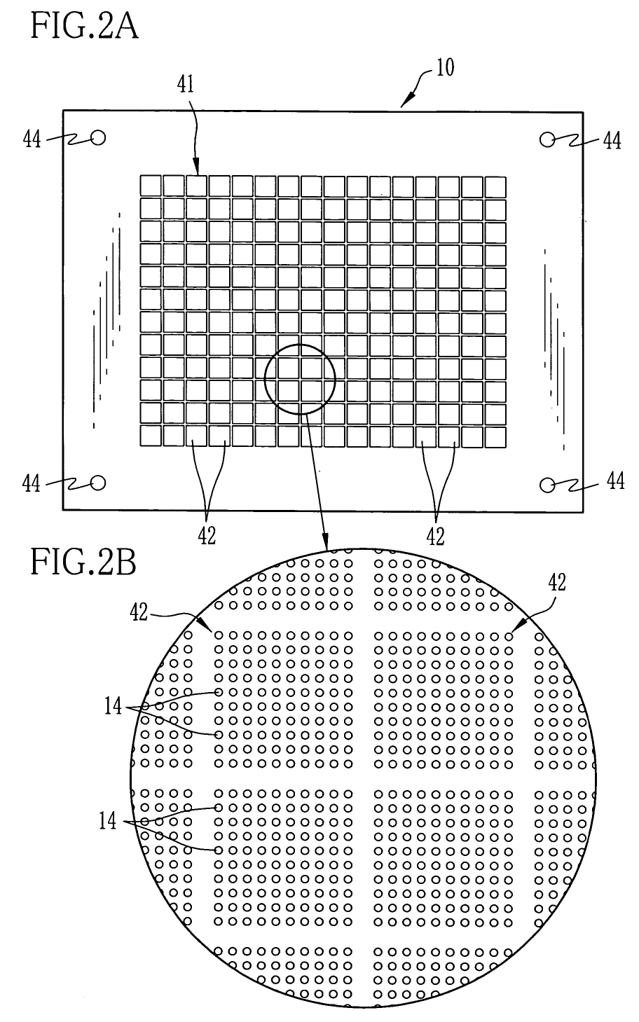

FIGS. 2A & 2B are explanatory views of the biochemical analysis unit 10. The substrate 11 is formed of the materials which can decrease the light intensity so as to prevent the scattering of the light, for example metal, ceramics, plastics, and the like. When the light does not scatter, it is prevented to misunderstand that the light would be generated from the other areas than the some spot areas from which the light is generated. When the materials having high efficiency for decreasing the light intensity is used, the misunderstanding is prevented, and the analysis data having high reliability is obtained. The rate of decreasing the intensity of the light generated from the one spot area becomes preferably at most ⅕, and especially at most 1/10 in the neighboring spot area.

The thickness of the substrate is preferably in the range of 50 to 1000 μm, and especially in the range of 100 to 500 μm. As the metals, there are copper, silver, gold, zinc, plumbum, aluminum, titanium, tin, chromium, iron, nickel, cobalt, tantalum and the like. Further, alloys, such as stainless, brass and the like, may be used. However, the metals are not restricted in them. Furthermore, as the ceramics, there are alumina, zirconia and the like. However, the materials to be used are not restricted in them.

As the plastics, there are olefins (for example, polyethylene, polypropylene, and the like), polystyrene, acryl resin (for example, polymethylmethacrylate, and the like), polymers containing chlorine (for example, polyvinyl chloride, polyvinylidene chloride and the like), polymers containing fluorine (for example, polychlorotrifluoroethylene, and the like), polycarbonates, polyesters, (for example, polyethylene naphthalate, polyethylene telephthalate and the like), polyamide (for example nylon-6, nylon-66 and the like), polyimide, polysulfonate, polyphenylen sulfide, silicon resins (for example, polydiphenyl cyclohexane and the like), phenol resins (for example, noborac and the like), epoxy resins, polyurethane, celluloses (for example, cellulose acetate, nitrocellulose and the like), and the like. Further, there are copolymers (for example butadiene-cellulose copolymer, and the like). Furthermore the above polymers may be blended. However, the sorts of the plastics are not restricted in them.

It is preferable to use the plastics as the materials of the substrate, since the through holes are easily formed. However, in this case, the light intensity is hardly decreased. In order to decrease the light intensity moreover, preferably, metal oxide particles or glass fiber particles are added to the plastics, and dispersed therein. As the metal oxide particles, there are silicon dioxide, alumina, titanium dioxide, iron oxide, cupper oxide and the like. However, the sorts of the metal oxide are not restricted in them.

A method of forming the through holes 12 are a punching method, pulse discharging method, etching method, and methods in which a laser beam (exima laser and YAG laser) is applied to the substrate. However, the method of forming the through holes is not restricted in them, and selected depending on the material of the substrate.

In order to make the density of the through holes 12 higher, the area of a opening of the each through hole is preferably less than 5 mm², particularly less than 1 mm², and especially less than 0.3 mm², more especially less than 0,01 mm², and most especially less than 0.001 mm². Further, when the through hole has a nearly circular shape, its diameter is preferably 200 μm to 300 μm.

A arrangement pitch P of the through holes 12 (a distance in centers of two neighboring through holes 12) is preferably 50 μm to 3000 μm, and a length of the nearest edges between the two neighboring through holes is preferably 10 μm to 1500 μm. Further, the number of the through holes 12 in a unit area is preferably at least 10/cm², particularly at least 100/cm², especially at least 500/cm², most especially 1000/cm² to 10000/cm².

In order to make the cleaning effect better, a surface treatment is made on the substrate in which the through holes 12 are formed. When metals and alloys (for example stainless and the like) are used as the materials of the substrate 11, the surface treatment is made in at least one of corona discharging method, plasma discharging method and an anodic oxidization method. In the surface treatment, a surface treatment layer is formed on the substrate 11, and is a layer of metal oxide having hydrophilic property by containing carbonyl groups and carboxyl groups.

After the surface treatment, an adhesive agent is applied to a surface of the substrate 11, on which the membrane 13 is pressed for the insertion into the through holes 12. The method of applying the adhesive agent is not restricted. However, it is preferably a method of a roller coating, a wire bar coating, a dip coating, a blade coating, an air knife coating or the like. As the adhesive agents, there are styrene-butadiene rubber and acrylonitril-butadiene rubber. However, it is not restricted in them. Note that the excess adhesive agent is scratched and removed by the bleade, or may be removed with use of a laser beam for preventing the generation of the impurities in the following process. Note that the processes of the surface treatment of the substrate and the application of the adhesive agent can be omitted.

After the application of the adhesive agents, the membrane 13 is pressed into the through holes 12. As the membrane 13, there are porous materials and fiber materials. Note that the porous materials and the fiber materials are used simultaneously. The membrane 13 used in the present invention may be one of the porous materials (organic, inorganic porous materials or mixture thereof), the fiber materials (organic or inorganic fiber materials). Further these may be mixed. The thickness of the membrane 13 is not restricted especially. However, it may be in the range of 100 μm to 200 μm (0.10 mm to 0.20 mm). Further, a void ratio C in volume is a percentage of a total volume of the voids to the appearance volume of the absorptive materials.

The sorts of the organic porous materials are not restricted especially. However, they are preferably polymers, for example, cellulose derivatives (for example, nitro cellulose, regenerated cellulose, cellulose acetate, cellulose acetate butyrate, and the like), aliphatic polyamides (for example, nylon-6, nylon-6,6, nylon 4,10, and the like), polyolefines (for example, polyethylene, polypropyrene), polymers containing chlorinate (for example, polyvinyl chloride, polyvinylidene chloride and the like), fluorine resins (for example, polyvinylidene floride, polytetrafluoride and the like), polycarbonate, polysulfone, alginic acid, and derivatives thereof (for example, calcium alginate, ion complex of alginic acid/polylysine, and the like) collagen, and the like. Further, the copolymer or the complexes (or mixture) of these polymers may be used. Note that porous nylon is preferably used in view of the water absorbing properties in the present invention.

The sorts of the inorganic porous materials are not restricted. However, they are preferably metal (for example, platinum, gold, iron, silver, nickel, aluminum, and the like), metal oxide (for example, alumina, silica, titania, zeolite, and the like), salts of metals (hydroxyapatite, calcium sulfate and the like) complexes of them, and the like. Further, porous carbon materials (activated carbon and the like) may be used.

Further, organic fiber materials and the inorganic fiber materials are not restricted in them. However, as the organic fiber materials, the cellulose derivatives, aliphatic polyamides and the like can be used, and as the inorganic fiber materials, glass fiber and metal fiber can be used. Note that in order to increase the strength of the membrane 13, the fiber materials insoluble to the solvent can be used, while the porous materials can be dissolve to the solvent.

The pressing of the membrane 13 is intermittently made from up and down sides by press plates in the situation that the substrate 11 and the membrane 13 are superimposed. Note that when the organic porous or fiber materials are used as the membrane 13, the press plates are heated so as to increase the temperature of the substrate 11. Thus the membrane 13 becomes softened, and easily pressed into the through holes 12 to form the spot area 14. Further, a roller may be used instead of the press plates.

A flow-through area 41 in which the spot areas 14 are arranged has a generally rectangular shape, and is regularly sectioned into rectangular blocks in which a predetermined number of the spot areas 14 is formed. The size of the substrate is, for example, 70 mm in length and 90 mm in width. An area of each block 42 is about 4 mm. The blocks are matrix-likely arranged, and the number in length is 12, and that in width is 16. The features of the flow-through areas (namely size and the number of the block, and the size and the pitch of the arrangement of each spot area 14) are determined, corresponding to the feature of the CCD image sensor 32. Positioning holes 44 are used for attachment of the biochemical analysis unit to the cartridge 28. Note that the substrate 11 is separated into the blocks having the predetermined number of the spot areas 14 in this embodiment. However this sectioning may not be made. For example, the spot areas 14 may be arranged all over the flow-through area 41.

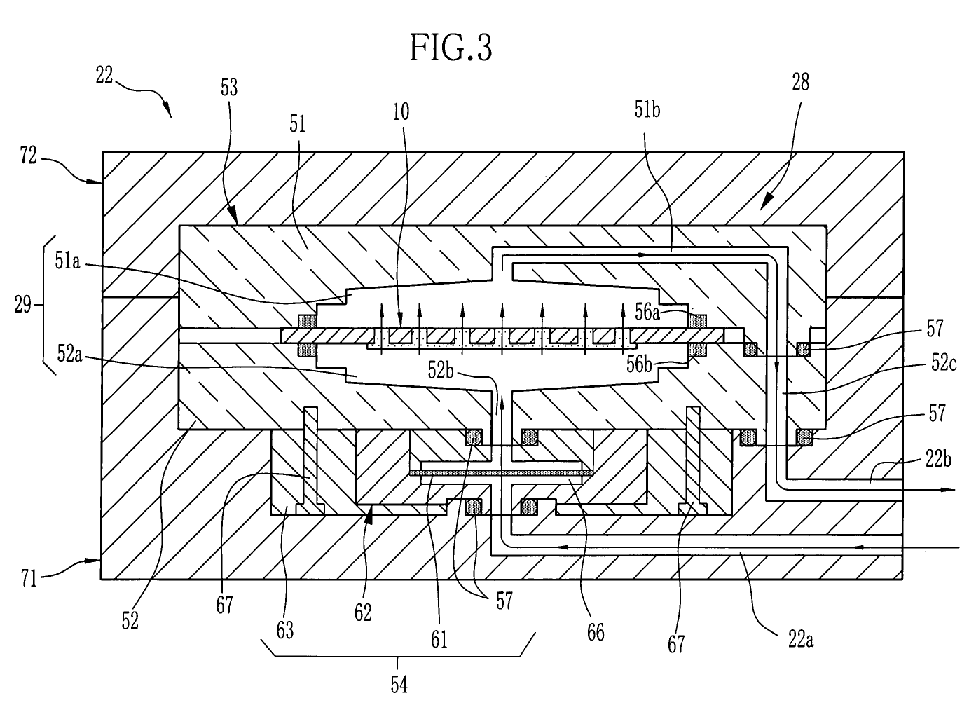

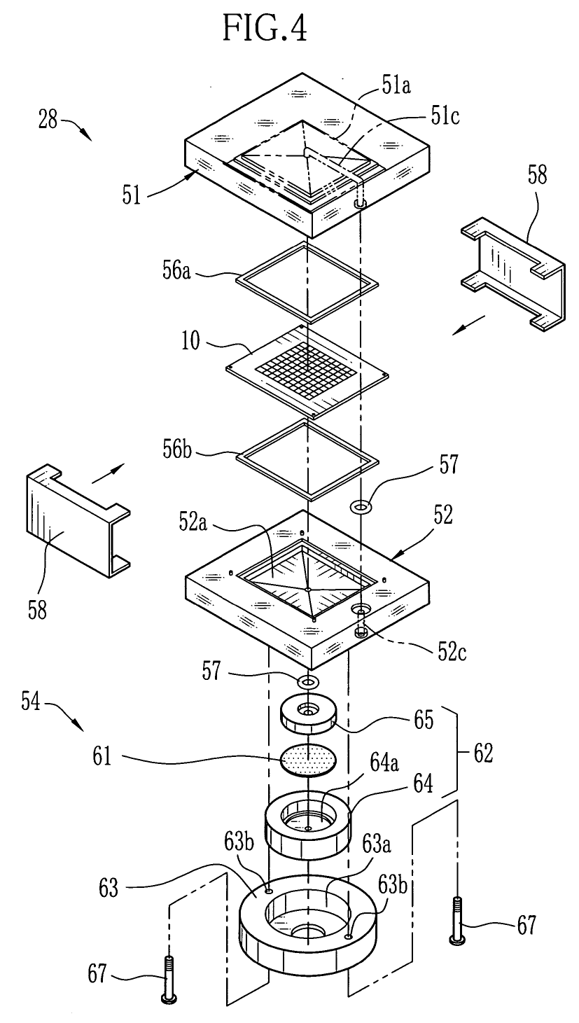

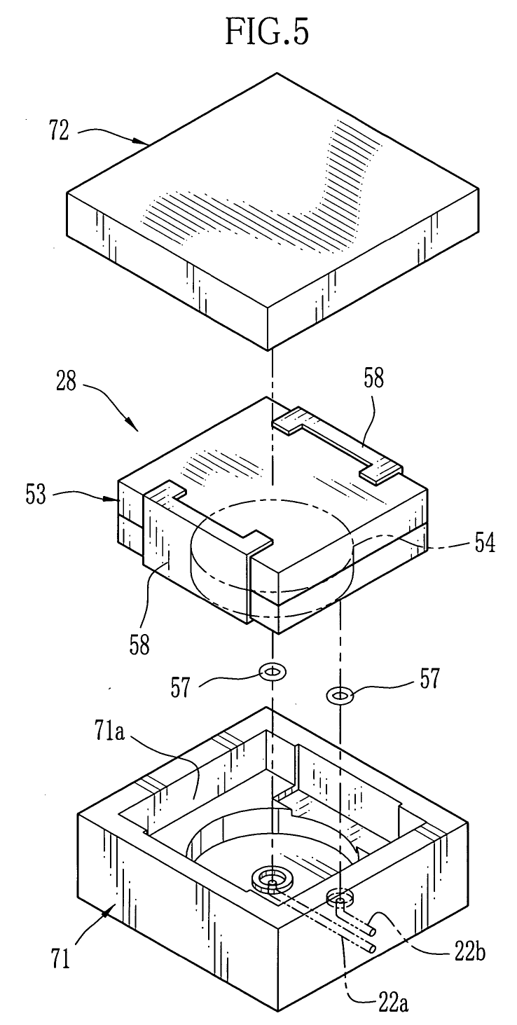

FIG. 3 is a sectional view of the cartridge 28 and the reactor main body 22 in which the cartridge 28 is loaded. FIGS. 4 & 5 are divisional perspective views of the cartridge 28 and the reactor respectively. As shown in FIGS. 3 & 4, the cartridge 28 is constructed of a cartridge main body 53 having an upper part 51 and a lower part 52, and a filter unit 54 which is fixedly attached to the cartridge main body 53 such that the filter 61 may be integrated with the cartridge main body 53. Recess 51a, 52a, each of which are nearly quadrangular pyramid shaped, are respectively formed on a bottom of the upper part 51 and a top of the lower part 52 in this figure. When the upper and lower parts 51, 52 are superimposed, the recesses 51a, 52a are combined to construct the chamber 29 whose section is rhombous shaped. The upper part 51 and the lower part 52 are formed from a transparent plastics, and the situation in the chamber can be recognized from outside.

The upper part 51 has an outlet path 51b for discharging the reaction solution from the chamber 29. The lower part 52 has an inlet path 52b for supplying the reaction solution into the chamber 29, and a connection path 52c for connecting the outlet path 51b to the discharge path 22b formed in the reactor 21.

In the lower part 52, positioning pins are provided at four corners of the recess 52a. The positioning pins are inserted into positioning holes 44 to make a poisoning of the biochemical analysis unit 10. After the positioning, the biochemical analysis unit 10 is covered with the upper part 52, and edge portions of the biochemical analysis unit 10 are sandwiched by the recesses 51a, 52a. Thus the spot areas 14 are positioned in the chamber 29. In edges of each recess 51a, 52a are provided packings 56a, 56b having a rectangular frame shape so as to surround the flow-through area 41 of the biochemical analysis unit 10. The packings 56a, 56b fill respective gaps between a biochemical analysis unit 10 and the upper part 51 and between the biochemical analysis unit 10 and the lower part 52. Thus the chamber 29 is tightly closed. A rubber made O-ring 57 as the packing is provided between the outlet path 51b and the connection path 52c to tightly close connected portions. Thus the upper part 51 and a lower part 52 containing the biochemical analysis unit 10 are fastened from both sides with two clamps 58 (see, FIGS. 4 & 5) having an almost U-shape in section.

The filter unit 54 is constructed of a filter 61, a filter case 62, and a retainer 63. The filter 61 is disposed between the supply path 22a and the inlet path 52b, so as to remove the foreign materials in the solution by filtration of the supplied reaction solution. The used filter 61, for example, is formed of a membrane 13 so as to have a disc-like shape.

The filter case 62 is constructed of a housing 64 and a lid 65. The housing 64 has a containing portion 64a in which the filter 61 is set and contained. After setting the filter 61, the lid 65 is fitted the containing portion 64a to cover the filter 61. Recesses are formed in an upper side of the housing 64 and a lower side of the lid 65 in this figure, and when the lid 65 is fitted to the housing 64, the filter 61 is disposed in a chamber 66 formed by the two recesses 51a, 52a. The chamber 66 is connected to the supply path 22a through a connection path formed in the housing 64, and connected to the inlet path 52b through an opening formed in the lid 65. The O-ring 57 for tightly-closing the passage is attached to a part for respectively connecting the inlet path and the supply path to the filter case 62. When supplied into the chamber 66, the reaction solution fills the chamber 66. Since the filter 61 is disposed in the chamber 66, the reaction solution flows over all area of the filter 61.

The retainer 63 supports the filter case 62 and fixes it to the cartridge main body 53. The retainer 63 has a containing portion 63a for containing the filter case 62 and a screw holes 63b into which screws 67 are inserted. The retainer 63 is fixedly attached to the cartridge main body 53 with the screws 67 in a situation that the filter case 62 is contained in the containing portion 63a. Thus the cartridge main body 53 and the filter 61 are integrated to construct the cartridge 28.

The reactor main body 22 is constructed of a housing 71 and a lid 72. The supply path 22a and the discharge path 22b are formed in the housing 71. On a side face of the housing 71, an entrance of the supply path 22a and an exit of the discharge path 22b are formed and connected to the circulating pipe 23. On a tope face of the housing 71, a load portion 71a on which the cartridge 28 is loaded is formed. The load portion 71a is formed corresponding to the shape of the cartridge 28. On the bottom of the load portion 71a, an exit of the supply path 22a and an entrance of the discharge path 22b are formed at such positions at which when the cartridge 28 is loaded, the supply path 22a is connected through a filter unit to the inlet path 52b and the discharge path 22b is connected to the connection path 52c. The exit of the supply path 22a and the entrance of the discharge path 22b are attached to the O-ring 57.

The lid 72 is disposed on the housing 71 so as to cover the loaded cartridge 28. In this situation, the lid 72 and the housing 71 are clamped by clamp members (not shown). Thus the upper part 51 and the lower part 52 of the cartridge 28 in the load portion 71a are pressed from upside and downside. Thus the tightness of the chamber 29 and connected parts of the paths are kept.

The operation of the above structure will be described below. In the spotting process, several sorts of probes are spotted and fixed to respective spot areas 14 of the biochemical analysis unit 10. The biochemical analysis unit 10 is set in the cartridge main body 53 of the cartridge 28 while the filter unit 54 is fixedly attached to the cartridge main body previously. Then the cartridge 28 is sent to the specific binding reaction process.

In the specific binding reaction process, the cartridge 28 is loaded in the reactor 22. Since the filter unit 54 is fixedly attached to the cartridge main body 53 in the cartridge 28, the biochemical analysis unit 10 and the filter 61 are simultaneously set to the reactor main body 22 in the loading operation. Therefore, the failure of loading the filter 61 and the loading mistake are prevented.

After the cartridge 28 is loaded, the valve 25 is opened and the reaction solution is supplied from the tank 26 to the chamber 29. The reaction solution flows through the circulation pipe 23 and the supply path 22a toward the filter unit 54. After the filtration with the filter 61, the reaction solution is supplied to the chamber 29. The chamber 29 and the circulation passage are filled with the reaction solution, and the air is extracted. In this situation, the valve 25 is closed.

When the pump 24 is driven in this situation, the reaction solution circularly flows through the circulation passage with application of the pressure. Since the filter 61 is disposed between the supply path 22a and the chamber 29, the reaction solution after the filtration with the filter 61 is supplied to the chamber 29. Therefore the clogging of the biochemical analysis unit 10 is prevented.

In the spot area 14 in which the probe having the complementary relation to the target is fixed, the flowage of the reaction solution causes the specific binding reaction. Otherwise, in the spot area in which the probe having no complementary relation to the target is fixed, the specific binding reaction does not occur.

When the specific binding reaction process is end, the valve 25 is opened such that the reaction solution in the circulation passage is discharged to the discharge vessel 27. Then, a cleaning liquid is fed into the circulation passage to make a cleaning. After the cleaning, the chemifluorescent reaction process is made, and thereafter the cartridge 28 is extracted from the reactor 21. Thereby the extraction of the filter 61 and the biochemical analysis unit 10 from the reactor 21 is made at the same time, since the filter unit 54 is fixedly attached to the cartridge main body 53. Accordingly, when the next experiment is made, the exchange of the filter 61 is not forgot. Further, the time for the operation becomes shorter than the case in which the filter and the biochemical analysis unit are separately exchanged.

The cartridge 28 removed from the reactor 21 is sent to the data reading process. In the data reading process, the biochemical analysis unit 10 is extracted from the cartridge 28 and the data reading is made with the detecting device 31. In the data reading process, the chemifluorescent materials as the labeling substances generate the light, which is received by the detecting device 31. Since the chemifluorescent materials remain in the spot area in which the specific binding reaction has been made, the spot area 14 in which the specific binding reaction has occurred generates a light, and the spot area 14 in which the specific binding reaction has not occurred does not generate a light. The biochemical analysis data is read by detecting the light with the detecting device 31. Since the filter 61 is provided, the undissolved compounds or the precipitations in the reaction solution are removed. Therefore the noise in the analysis data becomes smaller.

In this embodiment, the screws are used for fixing the filter unit to the cartridge main body. Thus the filter can be exchanged only when the screws are removed. Accordingly, the exchange of the filter in the cartridge made easily. When the easiness of the exchange of the filter is not considered, an adhesive agent may be used for fixing the filter case to the cartridge main body. Further, in this embodiment the filter is contained in the filter case other than the cartridge main body. However, the filter case may not be used, and a filter containing portion is formed in the cartridge main body, and the filter may be contained therein.

Various changes and modifications are possible in the present invention and may be understood to be within the present invention.

1. A cartridge for containing a biochemical analysis unit of flow-through type with arrangement of plural spot areas, said cartridge being removably contained in a reactor, said spot areas fixing probes which specifically react as reagents to a target as a test substance in a reaction solution, said cartridge comprising:
a cartridge main body for removably containing said biochemical analysis unit;
a chamber formed in said cartridge main body, said chamber having an inlet for supplying said reaction solution therein and an outlet for discharging said reaction solution, said biochemical analysis unit being positioned so as to partition said chamber into a supply part and a discharge part; and
a filter disposed upstream from said inlet, for filtering said reaction solution.

2. A cartridge as described in claim 1, further comprising a filter hold member for containing said filter, and said filter hold member is attached to said cartridge main body.

3. A cartridge as described in claim 2, wherein said filter hold member is removable from said cartridge main body such that said filter may be exchangeable, and a side for contacting said cartridge main body is open.

4. A cartridge as described in claim 2, wherein said cartridge main body is constructed of an upper part and a lower part, each of which has a form so as to divide said cartridge almost from a center of said chamber.

5. A cartridge as described in claim 4, wherein a face of said biochemical analysis unit is quadrangular and each of said supply part and said discharge part is quadrangular pyramid.

6. A cartridge as described in claim 4, further comprising a retention member for retaining a situation in which said biochemical analysis unit is sandwiched between said upper part and said lower part.
